(19) United States
(12) Patent Application Publication
(10) Pub. No.: US 2026/0123907 A1
(43) Pub. Date: May 7, 2026

(54) ULTRASONIC DIAGNOSTIC APPARATUS, LEARNING APPARATUS, AND IMAGE PROCESSING METHOD

(71) Applicant: CANON KABUSHIKI KAISHA, Tokyo (JP)

(72) Inventors: Shoya Sasaki, Kanagawa (JP); Naoya Iizuka, Kanagawa (JP); Kenichi Nagae, Kanagawa (JP)

(21) Appl. No.: 19/441,349

(22) Filed: Jan. 6, 2026

Related U.S. Application Data
(63) Continuation of application No. 17/153,351, filed on Jan. 20, 2021.

(30) Foreign Application Priority Data
Jan. 24, 2020 (JP) 2020-009950

(51) Int. Cl. A61B 8/06; A61B 8/00; G06N 20/00

(52) U.S. Cl. CPC A61B 8/06; A61B 8/4444; A61B 8/461; A61B 8/5246; G06N 20/00

(57) ABSTRACT

An ultrasonic diagnostic apparatus, comprising: an ultrasonic probe configured to transmit and receive ultrasonic waves to and from an object; and an estimation calculating unit configured to estimate data based on blood flow information from third data based on a received signal for image generation received by the ultrasonic probe by using a model having been machine-learned from learning data including first data based on a received signal for image generation that is obtained from an observation region and second data based on blood flow information of the observation region.

### CROSS REFERENCE OF RELATED APPLICATIONS

This application is a continuation of U.S. patent application Ser. No. 17/153,351, filed on Jan. 20, 2021, which claims the benefit of Japanese Patent Application No. 2020-009950, filed on Jan. 24, 2020, all of which is hereby incorporated by reference herein in its entirety.

### BACKGROUND

### Field of the Disclosure

The present disclosure relates to an ultrasonic diagnostic apparatus, a learning apparatus, and an image processing method and, in particular, to a technique for improving image quality of an ultrasonic diagnostic apparatus.

### Description of the Related Art

Ultrasonic diagnostic apparatuses are widely used in clinical practice as image diagnostic apparatuses due to, for example, simplicity, high resolution performance, and real-time performance thereof. A general method of generating an ultrasonic image includes beamforming of a transmit beam and phasing addition processing of a received signal. Beamforming of a transmit beam is performed by inputting a voltage waveform provided with a time delay relative to a plurality of conversion elements and causing ultrasonic waves to converge inside a living organism. Phasing addition of a received signal is performed by receiving ultrasonic waves reflected by a structure inside a living organism by a plurality of conversion elements, and providing to obtained received signals a time delay in consideration of a path length with respect to a point of interest, and then adding up the received signals. Due to the beamforming of the transmit beam and the phasing addition processing, reflected signals from the point of interest are selectively extracted to perform imaging. By performing control so that the inside of an imaging region is scanned by the transmit beam, it is possible to obtain an image of a region desired to be observed.

In such ultrasonic diagnostic apparatuses, the Doppler method in which blood flow information is imaged using the Doppler effect is widely used. One such Doppler method is the color Doppler method. In the color Doppler method, transmission/reception of an ultrasonic pulse is performed a plurality of times on a same scan line and a phase difference (an amount of Doppler shift) of a component derived from blood flow is extracted from received signals. The extraction of an amount of Doppler shift is performed by applying an MTI (Moving Target Indicator) filter to received signals at a same position but of different time series, and reducing components (clutter components) derived from tissue with small movement. Blood flow information (Doppler information) such as a velocity and a dispersion of blood flow is obtained from the extracted component derived from the blood flow.

Japanese Patent Application Laid-open No. H01-153144 discloses the Doppler method using an MTI filter. Japanese Patent Application Laid-open No. 2019-25044 discloses a medical imaging apparatus using a restorer constituted by a neural network.

### SUMMARY

A maximum velocity that can be acquired by the color Doppler method is known to be constrained by a repetition frequency of an ultrasonic pulse. Since a component with a frequency higher than the repetition frequency causes aliasing when calculating a phase difference, the component becomes indistinguishable from a component with a low frequency. For example, since the observation of a deep part requires lowering of the repetition frequency, there is a limit to velocities that can be acquired.

In addition, in the color Doppler method, blood flow information is displayed by being superimposed on a normal B-mode image. Therefore, in addition to transmission/reception of an ultrasonic pulse for creating a normal B-mode image, transmission/reception of an ultrasonic pulse for a color Doppler image also has to be performed. As a result, a frame rate drops more in a normal B-mode. Furthermore, while the number of transmissions/receptions of an ultrasonic pulse on a same scan line may be increased in order to improve color Doppler accuracy, this causes a further drop in the frame rate.

The present disclosure has been proposed in consideration of the problem described above and an object thereof is to provide an ultrasonic diagnostic apparatus that enables blood flow information (Doppler information) of a wide range to be obtained while reducing an effect of a drop in a frame rate.

The disclosure includes an ultrasonic diagnostic apparatus, comprising: an ultrasonic probe configured to transmit and receive ultrasonic waves to and from an object; and an estimation calculating unit configured to estimate data based on blood flow information from third data based on a received signal for image generation received by the ultrasonic probe by using a model having been machine-learned from learning data including first data based on a received signal for image generation that is obtained from an observation region and second data based on blood flow information of the observation region.

The disclosure further includes a learning apparatus performing machine learning of a learning model to be used by the estimation calculating unit of the ultrasonic diagnostic apparatus according to claim 1, the learning apparatus comprising a learning unit that performs machine learning of the learning model by using learning data that includes data, based on a received signal of a reflected ultrasonic wave obtained from an observation region, as input data and blood flow information, extracted from a reflected ultrasonic wave obtained by scanning the observation region a plurality of times, as correct answer data.

The disclosure further includes an image processing method comprising: a receiving step of transmitting an ultrasonic wave to an object and receiving a reflected ultrasonic wave from the object by using an ultrasonic probe; an estimation calculating step of estimating data based on the blood flow information from third data based on a received signal for image generation received in the receiving step by using a learning model having been machine-learned using learning data including first data based on a received signal for image generation that is obtained from an observation region and second data based on blood flow information of the observation region; and a displaying step of displaying on a display apparatus an image based on data estimated in the estimation calculating step.

The disclosure still further includes a computer-readable medium non-transitorily storing a program for causing a processor to execute the respective steps of the above-described image processing method.

According to the an ultrasonic diagnostic apparatus of the present disclosure, blood flow information (Doppler information) of a wide range can be obtained with reducing an effect of a drop in a frame rate.

Further features of the present invention will become apparent from the following description of exemplary embodiments with reference to the attached drawings.

### BRIEF DESCRIPTION OF THE DRAWINGS

FIG. 1 is a block diagram showing an example of a configuration of an ultrasonic diagnostic apparatus;

FIG. 2 is a block diagram showing an example of functions included in a received signal processing block according to a first embodiment;

FIG. 3 is a diagram showing an example of a learning apparatus for learning a learning model;

FIG. 4 is a diagram for explaining learning data;

FIG. 5 is a diagram showing an example of a GUI for creating learning data;

FIGS. 6A and 6B are diagrams representing a time sequence of image generation processing;

FIG. 7 is a diagram showing a flow of image generation and display processing; and

FIGS. 8A to 8C are diagrams representing an example of display by a display apparatus.

### DESCRIPTION OF THE EMBODIMENTS

### First Embodiment

A first embodiment of the present invention will be described. In the present embodiment, blood flow information is estimated from a plurality of frames'worth of a received signal for B-mode image generation. A learned model having been machine-learned is used for the estimation. Since the number of times a received signal for Doppler image generation is acquired can be reduced, an image corresponding to blood flow information can be displayed in a state of a higher frame rate than displaying a normal color Doppler image. In addition, since blood flow information is obtained by estimation, a maximum blood flow velocity that can be acquired is not constrained by the repetition frequency. Accordingly, a low-velocity blood flow and a high-velocity blood flow which are difficult to display with a normal color Doppler method can be displayed at the same time.

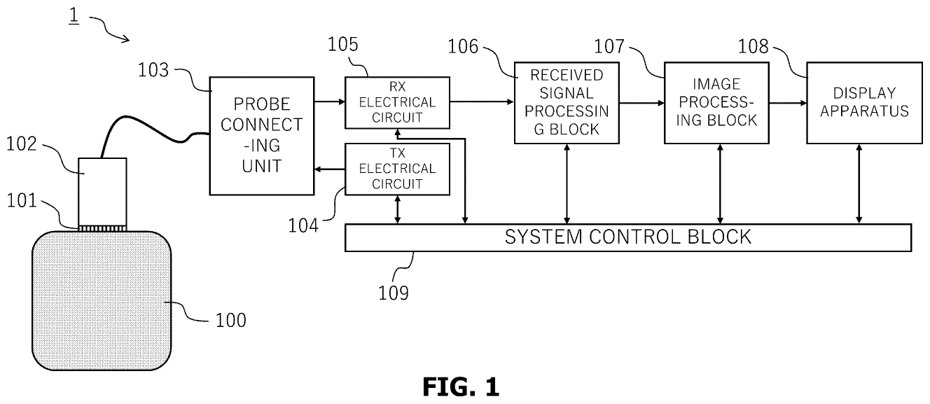

FIG. 1 is a block diagram showing an example of a hardware configuration of an ultrasonic diagnostic apparatus 1 according to the present embodiment. In general, the ultrasonic diagnostic apparatus 1 has an ultrasonic probe (an ultrasonic transducer) 102, a probe connecting unit 103, a transmission electrical circuit 104, a reception electrical circuit 105, a received signal processing block 106, an image processing block 107, a display apparatus 108, and a system control block 109. The ultrasonic diagnostic apparatus 1 is a system for transmitting an ultrasonic pulse to an object 100 from the ultrasonic probe 102, receiving reflected ultrasonic waves having been reflected inside the object 100, and generating image information (an ultrasonic image) of the inside of the object 100. The ultrasonic image obtained by the ultrasonic diagnostic apparatus 1 is to be used in various clinical examinations.

The ultrasonic probe 102 is a probe adopting an electronic scan system and has a plurality of transducers 101 arranged one-dimensionally or two-dimensionally at a tip thereof. The transducer 101 is an electric mechanical conversion element that performs mutual conversion between an electric signal (a voltage pulse signal) and an ultrasonic wave (an acoustic wave). The ultrasonic probe 102 transmits ultrasonic waves from the plurality of transducers 101 to the object 100 and receives reflected ultrasonic waves from the object 100 by the plurality of transducers 101. Reflected acoustic waves reflect a difference in acoustic impedances inside the object 100.

The transmission electrical circuit 104 is a transmitting unit that outputs a pulse signal (a drive signal) with respect to the plurality of transducers 101. By applying a pulse signal with a time difference with respect to the plurality of transducers 101, ultrasonic waves with different delay times are transmitted from the plurality of transducers 101 and a transmission ultrasonic beam is formed. By selectively changing the transducer 101 to which the pulse signal is applied (in other words, the transducer 101 to be driven) and changing a delay time (an application timing) of the pulse signal, a direction and a focus of the transmission ultrasonic beam can be controlled. An observation region inside the object 100 is scanned by sequentially changing the direction and the focus of the transmission ultrasonic beam. By transmitting a pulse signal with a prescribed driving waveform to the transducers 101, the transmission electrical circuit 104 generates a transmission ultrasonic wave having a prescribed transmission waveform in the transducers 101. The reception electrical circuit 105 is a receiving unit that inputs, as a received signal, an electric signal output from the transducer 101 having received a reflected ultrasonic wave. The received signal is input to the received signal processing block 106.

Operations of the transmission electrical circuit 104 and the reception electrical circuit 105 or, in other words, transmission/reception of ultrasonic waves is controlled by the system control block 109. The system control block 109 changes a position where a voltage signal or a transmission ultrasonic wave is formed in accordance with, for example, respective generation of a B-mode image and a Doppler image to be described later.

When generating a B-mode image, a received signal of a reflected ultrasonic wave obtained by scanning an observation region is acquired and used for image generation. A received signal corresponding to one frame's worth of a B-mode image are obtained by one scan of the observation region. When generating a Doppler image, a received signal of a reflected ultrasonic wave obtained by performing transmission/reception of an ultrasonic wave a plurality of times on each of a plurality of scan lines in the observation region is acquired and used for image generation or, in other words, extraction of blood flow information. A scan for Doppler image generation may be performed by a system in which transmission/reception is performed a plurality of times on one scan line and then transmission/reception is performed on a next scan line or a system in which an operation of performing one transmission/reception on each scan line is repeated a plurality of times. An observation region of a Doppler image is usually a part of an observation region of a B-mode image. In addition, transmission/reception of an ultrasonic wave for B-mode image generation and transmission/reception of an ultrasonic wave for Doppler image generation are usually alternately performed.

In the present specification, both an analog signal output from the transducer 101 and digital data obtained by sampling (digitally converting) the analog signal will be referred to as a received signal without particular distinction. However, a received signal will sometimes be described as received data depending on the context in order to clearly indicate that the received signal is digital data.

The received signal processing block 106 is an image generating unit that generates image data based on a received signal obtained from the ultrasonic probe 102. The image processing block 107 applies image processing such as brightness adjustment, interpolation, and filter processing on the image data generated by the received signal processing block 106. The display apparatus 108 is a display unit for displaying image data and various kinds of information and is constituted by, for example, a liquid crystal display or an organic EL display. The system control block 109 is a control unit that integrally controls the transmission electrical circuit 104, the reception electrical circuit 105, the received signal processing block 106, the image processing block 107, the display apparatus 108, and the like.

### Configuration of Received Signal Processing Block

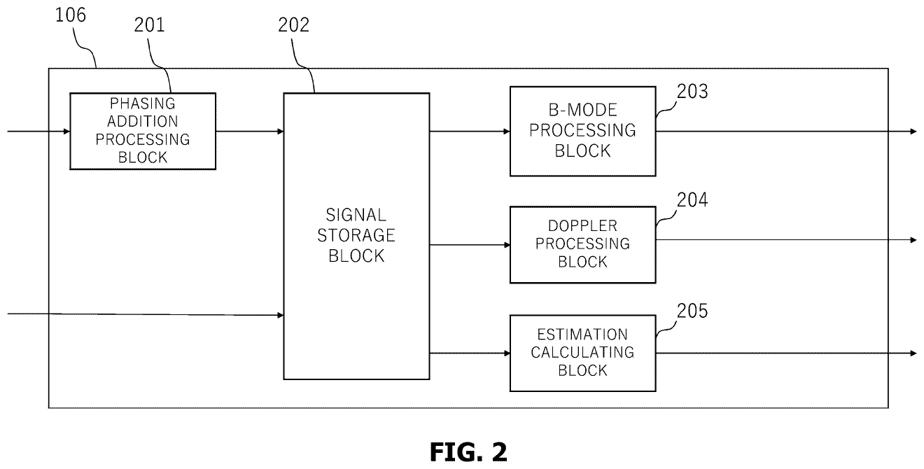

FIG. 2 is a block diagram showing an example of functions included in the received signal processing block 106. The received signal processing block 106 has a phasing addition processing block 201, a signal storage block 202, a B-mode processing block 203, a Doppler processing block 204, and an estimation calculating block 205.

The phasing addition processing block 201 performs phasing addition and quadrature detection processing on the received signal obtained by the reception electrical circuit 105 and saves the processed received signal in the signal storage block 202. Phasing addition processing refers to processing for forming a reception ultrasonic beam by varying a delay time for each transducer 101 and adding up received signals of the plurality of transducers 101 and is also called Delay and Sum (DAS) beamforming. Quadrature detection processing refers to processing for converting a received signal into an in-phase signal (an I signal) and a quadrature signal (a Q signal) of a baseband. The phasing addition processing and the quadrature detection processing are performed by the phasing addition processing block based on an element arrangement and various conditions of image generation (aperture control and signal filtering) that are input from the system control block 109. After being subjected to the phasing addition processing and the quadrature detection processing, the received signal for B-mode image generation is saved in the signal storage block 202. In addition, the received signal for Doppler image generation is saved in the signal storage block 202.

The B-mode processing block 203 performs envelope detection processing, logarithmic compression processing, and the like on the received signal for B-mode image generation that is saved in the signal storage block 202 and generates image data in which signal strength at each point inside the observation region is expressed by brightness intensity.

The Doppler processing block 204 extracts blood flow information (Doppler information) by a method to be described later from the received signal for Doppler image generation that is saved in the signal storage block 202 and generates blood flow image data that represents imaged blood flow information. The Doppler processing block 204 corresponds to the Doppler processing unit according to the present invention.

The estimation calculating block 205 (an estimation calculating unit) uses a model to estimate data based on blood flow information from third data based on a received signal for image generation having been received by an ultrasonic probe. In the present embodiment, the estimation calculating block 205 generates (estimates) estimated blood flow information data (fourth data) based on a received signal for B-mode image generation that is saved in the signal storage block 202. The estimation calculating block 205 has a learned model having been machine-learned in advance so as to output blood flow information using a received signal for B-mode image generation as an input, and generates (estimates) estimated blood flow information data using the learned model. The estimation calculating block 205 corresponds to the estimation calculating unit according to the present invention.

Image data output from the B-mode processing block 203, the Doppler processing block 204, and the estimation calculating block 205 is subjected to processing by the image processing block 107 and finally displayed by the display apparatus 108. A blood flow image may be displayed by being superimposed on a B-mode image or displayed without being superimposed on a B-mode image. Hereinafter, an image including blood flow information will be referred to as a color Doppler image or simply referred to as a Doppler image.

The received signal processing block 106 may be constituted by one or more processors and a memory. In this case, functions of the respective blocks 201 to 205 shown in FIG. 2 are to be realized by a computer program. For example, the functions of the respective blocks 201 to 205 can be provided by having a CPU load and execute a program stored in the memory. Other than the CPU, the received signal processing block 106 may include a processor (a GPU, an FPGA, or the like) responsible for operations of the B-mode processing block 203 and operations of the estimation calculating block 205. In particular, an FPGA is effectively used in the B-mode processing block 203 to which a large amount of data is input at the same time and a GPU is effectively used when executing operations in an efficient manner as in the estimation calculating block 205. The memory favorably includes a memory for storing a program in a non-transitory manner, a memory for temporarily saving data such as a received signal, and a working memory to be used by the CPU.

### Doppler Processing Block

The Doppler processing block 204 extracts blood flow information based on the Doppler effect of an object inside a scan range by performing a frequency analysis of a received signal for Doppler image generation that is saved in the signal storage block 202. While an example in which the object is blood will be mainly described in the present embodiment, alternatively, the object may be an object such as internal tissue or a contrast agent. In addition, an example of blood flow information includes at least any of a velocity, a dispersion value, and a power value. Furthermore, the Doppler processing block 204 may obtain blood flow information at one point (one position) in the object or obtain blood flow information at a plurality of positions in a depth direction. Moreover, the Doppler processing block 204 may obtain an average velocity or a maximum velocity in a prescribed depth range and, further, obtain velocities at a plurality of time points in a time series so that a time variation of velocities can be displayed.

Due to the Doppler processing block 204, the ultrasonic diagnostic apparatus 1 according to the present embodiment can execute a color Doppler method that is also known as a Color Flow Mapping (CFM) method. In the CFM method, transmission/reception of an ultrasonic wave is performed a plurality of times on each of a plurality of scan lines. The Doppler processing block 204 extracts a component derived from blood flow by applying an MTI (Moving Target Indicator) filter with respect to received data at a same position to reduce components derived from tissue with small movement (clutter components). In addition, blood flow information such as a velocity of blood flow, a dispersion of blood flow, and power of blood flow are calculated from the blood flow component. The display apparatus 108 (to be described later) displays blood flow information (blood flow image data) that represents a calculation result in color in two-dimensions by superimposing the blood flow information on B-mode image data.

### Estimation Calculating Block

The estimation calculating block 205 will be described. The estimation calculating block 205 performs processing for estimating blood flow information (Doppler image data) using a learned model. The learned model is machine-learned so as to estimate data based on movement information of the observation region from data based on a received signal of a reflected ultrasonic wave that is obtained from a prescribed scan range. More specifically, in the present embodiment, the learning model is learned so that, when data obtained by applying phasing addition processing to a plurality of frames'worth of a received signal obtained by scanning the observation region a plurality of times in order to generate a B-mode image is input to the learning model, the learning model outputs blood flow information data in the same observation region.

The model is machine-learned using learning data that includes first data (input data) based on a received signal for image generation that is obtained from the observation region and second data (correct answer data) based on the observation region. Examples of a specific algorithm for machine learning include a nearest neighbor method, a naive Bayes method, and a support vector machine. Another example is deep learning that autonomously generates a feature amount and a coupling weight coefficient for learning using a neural network. A usable algorithm among those described above can be appropriately used and applied to the present embodiment.

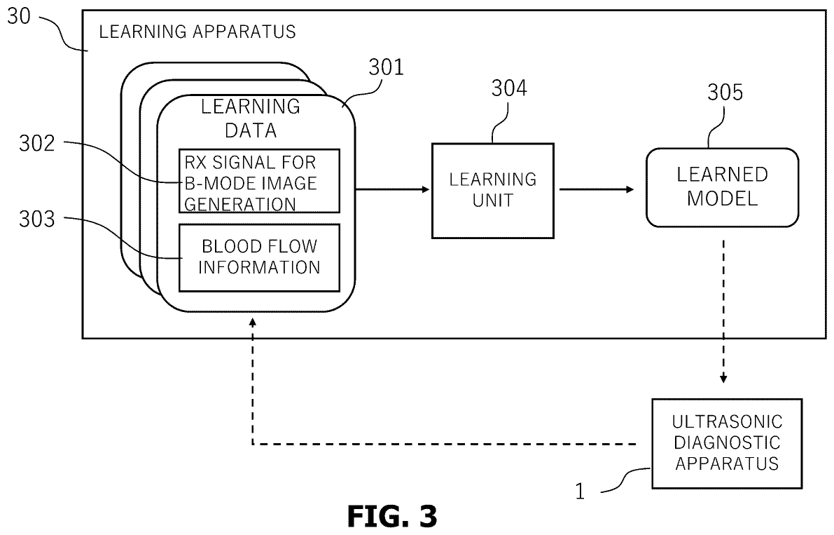

FIG. 3 shows an example of a learning apparatus 30 that performs machine learning of a model. The learning apparatus 30 has a learning unit (a learner) 304 that carries out machine learning of a model using a plurality of pieces of learning data 301. The learning unit 304 may use any of the machine learning algorithms exemplified above or may use another machine learning algorithm. The learning data 301 is constituted by a pair of input data and correct answer data (teacher data). In the present embodiment, a received signal 302 for B-mode image generation is used as input data and blood flow information 303 acquired using the color Doppler method is used as correct answer data. The learning unit 304 learns a correlation between the received signal 302 and the blood flow information 303 based on the plurality of pieces of supplied learning data 301 and creates a learned model 305. Accordingly, the learned model 305 can acquire a function (a capability) of generating blood flow information as output data when a received signal for B-mode image generation is given as input data. The learned model 305 is mounted to a program to be executed by the estimation calculating block 205 of the ultrasonic diagnostic apparatus 1. Learning of a model (generation processing of the learned model 305) is desirably performed before being incorporated into the ultrasonic diagnostic apparatus 1. However, when the ultrasonic diagnostic apparatus 1 has a learning function, learning (new learning or additional learning) may be performed using image data obtained by the ultrasonic diagnostic apparatus 1.

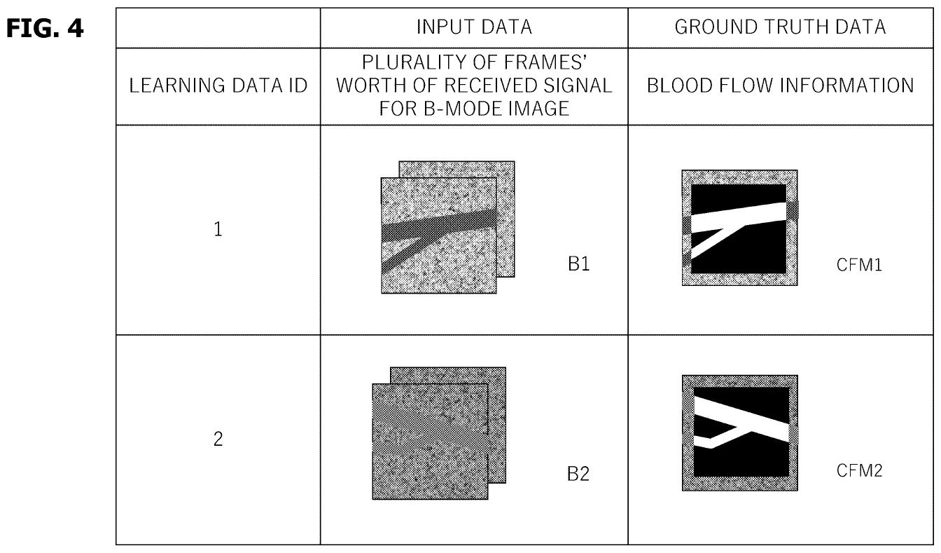

The learning data will now be described in greater detail with reference to FIG. 4. The input data included in the learning data is a plurality of frames'worth of a received signal for B-mode image generation of a given object. In addition, the correct answer data is blood flow information that is obtained by imaging the same object using the color Doppler method.

FIG. 4 exemplifies two pieces of learning data ID1 and ID2. The input data of the learning data ID1 is two frames'worth of a received signal B1 for B-mode image generation. In addition, the correct answer data of the learning data ID1 is blood flow information CFM1 obtained by imaging the same object using the color Doppler method. While the observation region of the received signal for B-mode image generation and the observation region of the blood flow information are desirably the same, a part of the observation region of the received signal for B-mode image generation may constitute the observation region of the blood flow information. In this case, a range corresponding to the observation region of blood flow information is cut out from the received signal for B-mode image generation and used as learning data (input data).

In addition, the input data of the learning data ID2 is two frames'worth of a received signal B2 for B-mode image generation acquired using an object that differs from the object of the learning data ID1 as an object. The correct answer data of the learning data ID2 is blood flow information CFM2 obtained by imaging the same object as the received signal B2 using the color Doppler method. While two frames'worth of a received signal for B-mode image generation is used as input data in this case, three frames'worth or more of a received signal may be used as input data or one frame's worth of a received signal may be used as input data.

Performing learning using learning data acquired under various conditions enables learning to be performed with respect to input of various patterns, and an image with good image quality can be expected to be estimated even during actual use. Therefore, a received signal for B-mode image generation and blood flow information are preferably acquired under different conditions with respect to a same object. It should be noted that, as an object, any of a digital phantom that can be imaged by a transmission/reception simulation of ultrasonic waves, an actual phantom, and an actual living organism may be used.

While an example in which input data of learning data is a plurality of frames'worth of a received signal for B-mode image generation is described in the present embodiment, the input data may further include acquisition conditions (imaging conditions) of the received signal for B-mode image generation. Examples of imaging conditions include a wavefront shape of a transmission ultrasonic wave, a transmission frequency of the transmission ultrasonic wave, a band of a bandpass filter, a type and/or a portion of an object, and a contact angle of the ultrasonic probe 102 relative to a body axis. Examples of the wavefront shape of a transmission ultrasonic wave include a convergent beam, a plane wave, and a diffuse wave. Including information regarding a transmission ultrasonic wave in the input data enables estimation in accordance with an ultrasonic wave used to acquire a received signal for B-mode image generation to be performed and improves estimation accuracy. In addition, including information regarding the object or information regarding the contact angle of a probe in the input data enables estimation in accordance with a feature of each site to be performed and a further increase in estimation accuracy is expected. Examples of a feature of each site include the presence of a surface fat layer, the presence of a high brightness region created by a fascial structure, and the presence of a low brightness region due to a thick blood vessel. The input data may further include information such as a field of medicine, gender, BMI, age, and a pathological condition and, accordingly, there is a possibility that a learned model corresponding to further detailed conditions can be obtained and a further increase in estimation accuracy is expected.

In addition, the learned model 305 of the estimation calculating block 205 mounted to the ultrasonic diagnostic apparatus 1 may be a model having learned image data of all fields of medicine or a model having learned image data of each field of medicine. When a model having learned image data of each field of medicine is mounted, the system control block 109 may cause the user of the ultrasonic diagnostic apparatus 1 to input or select information regarding a field of medicine to change the learned model to be used in accordance with the field of medicine. It is expected that estimation accuracy will further increase by selectively using a model for each field of medicine in which imaging sites are limited to a certain degree.

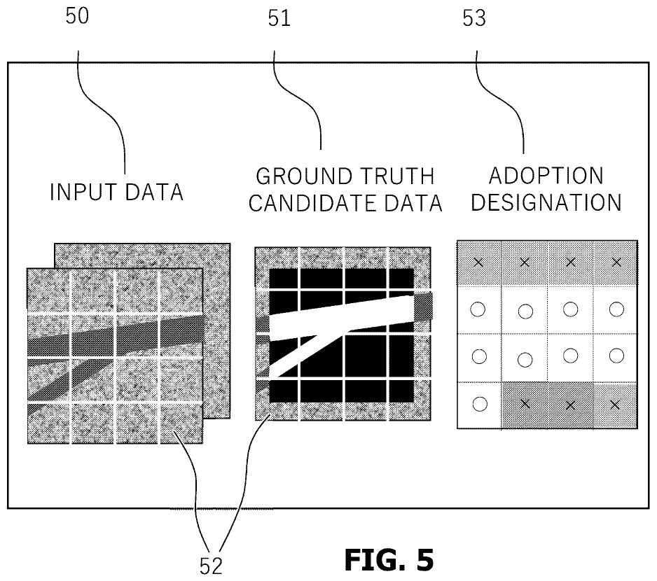

In learning, preprocessing of input data and correct answer data may be further performed using a GUI such as that shown in FIG. 5. Input data 50 and correct answer candidate data 51 are shown in a display screen, and indicators 52 that divide each piece of data into a plurality of regions are displayed. In the example shown in FIG. 5, images are divided into 16 regions in a 4 by 4 arrangement. An adoption designation box 53 is a user interface that enables a user to designate whether to adopt or reject each region. The user enters “o” into a region to be adopted as learning data and “x” into a region to be excluded while comparing the input data 50 and the correct answer candidate data 51 with each other. Accordingly, regions not suitable for learning such as a region that does not include blood flow information and a region where unexpected image deterioration has occurred in the correct answer candidate data 51 can be excluded. While FIG. 4 has been described on the assumption that an entire image is to be used as one piece of image data, when an image is divided into a plurality of regions as shown in FIG. 5, an image (a partial image) of each of the regions is used as one piece of learning data. In this case, the learning model accepts an image of a same size (resolution) as the input data 50 as input and outputs an image of a same size as the correct answer candidate data 51. In the example shown in FIG. 5, since there are 9 regions to be adopted, 9 sets of learning data are to be generated.

The learned model 305 obtained by performing machine learning using such imaging conditions and a received signal for B-mode image generation as input data and blood flow information as correct answer data operates on the estimation calculating block 205. Consequently, the estimation calculating block 205 is expected to estimate blood flow information from the input imaging conditions and the input received signal for B-mode image generation and output the estimated blood flow information.

### Image Generation Method

Next, details of processing for image generation according to the present embodiment will be described with reference to FIG. 1. When an imaging instruction is input from a GUI (not illustrated), the system control block 109 having received the instruction from the GUI inputs a transmission instruction of ultrasonic waves to the transmission electrical circuit 104. The transmission instruction favorably includes a parameter for calculating a delay time and sound velocity information. Based on the transmission instruction from the system control block 109, the transmission electrical circuit 104 outputs a plurality of voltage waveforms having a delay time to the plurality of transducers 101 of the ultrasonic probe 102 through the probe connecting unit 103. In the present embodiment, a transmission ultrasonic wave is a convergent beam and an imaging range is to be scanned by the transmission ultrasonic wave.

The transmission ultrasonic waves having been transmitted from the plurality of transducers 101 propagate inside the object and create a reflected ultrasonic wave that reflects a difference in acoustic impedances inside the object. The reflected ultrasonic wave is received by the plurality of transducers 101 and converted into a voltage waveform (a voltage signal). The voltage waveform is input to the reception electrical circuit 105 through the probe connecting unit 103. The reception electrical circuit 105 amplifies and digitally samples the voltage waveform as necessary and outputs the voltage waveform as a received signal to the received signal processing block 106. One frame's worth of a received signal for B-mode image generation is obtained by scanning a B-mode imaging range with a convergent beam. A received signal for Doppler image generation is obtained by performing transmission/reception of an ultrasonic wave a plurality of times on each of a plurality of scan lines in a Doppler image imaging range.

The received signal processing block 106 performs one of or both of phasing addition processing and quadrature detection processing on a received signal. With respect to a received signal for B-mode image generation obtained by the reception electrical circuit 105, the phasing addition processing block 201 performs phasing addition based on an element arrangement and various conditions (aperture control, signal filtering) of image generation that are input from the system control block 109. The received signal processing block 106 further saves the signal subjected to the phasing addition and quadrature detection processing in the signal storage block 202. The signal is transmitted to the B-mode processing block 203. The B-mode processing block 203 performs envelope detection processing, logarithmic compression processing, and the like and generates B-mode image data in which signal strength at each point inside the observation region is expressed by brightness intensity.

In a similar manner, the received signal for Doppler image generation obtained by the reception electrical circuit 105 is saved in the signal storage block 202. The Doppler processing block 204 calculates blood flow information image data using the received signal for Doppler image generation.

The estimation calculating block 205 uses a plurality of frames'worth of the received signal for B-mode image generation as input to output estimated blood flow information data. Specifically, the estimation calculating block 205 acquires and outputs, as blood flow information data corresponding to the received signal, blood flow information obtained by inputting a plurality of frames'worth of the received signal for B-mode image generation to the learned model 305.

The B-mode image data, the blood flow information image data, and the estimated blood flow information data are input to the image processing block 107, and after being subjected to brightness adjustment, interpolation, and other filtering, the pieces of data are displayed by the display apparatus 108. Hereinafter, an image based on blood flow information image data having been generated by the Doppler processing block 204 or image data in which the blood flow information image data and a B-mode image are superimposed on each other will also be referred to as a normal Doppler image. In addition, an image based on image data based on estimated blood flow information image data having been estimated by the estimation calculating block 205 or image data in which the image data based on estimated blood flow information image data and a B-mode image are superimposed on each other will also be referred to as a pseudo-Doppler image or an estimated image.

Next, a control example of generation and display of an image in the ultrasonic diagnostic apparatus 1 will be described. The ultrasonic diagnostic apparatus 1 has at least any of the following three display modes. A first display mode is a mode in which a display image is updated using a normal Doppler image without using a pseudo-Doppler image. A second display mode is a mode in which a display image is updated using both a normal Doppler image and a pseudo-Doppler image. A third display mode is a mode in which a display image is updated using a pseudo-Doppler image without using a normal Doppler image. When the ultrasonic diagnostic apparatus 1 has a plurality of display modes, for example, a user is favorably able to switch among the display modes.

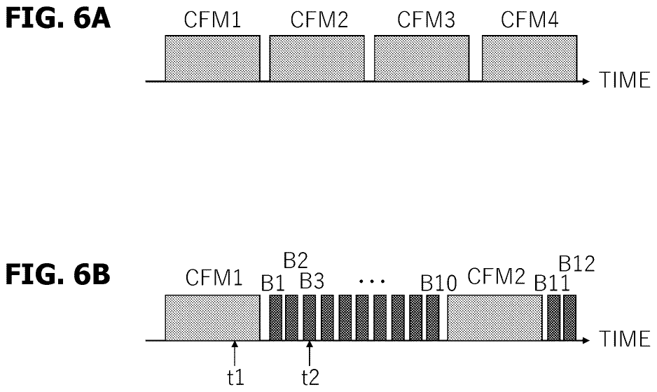

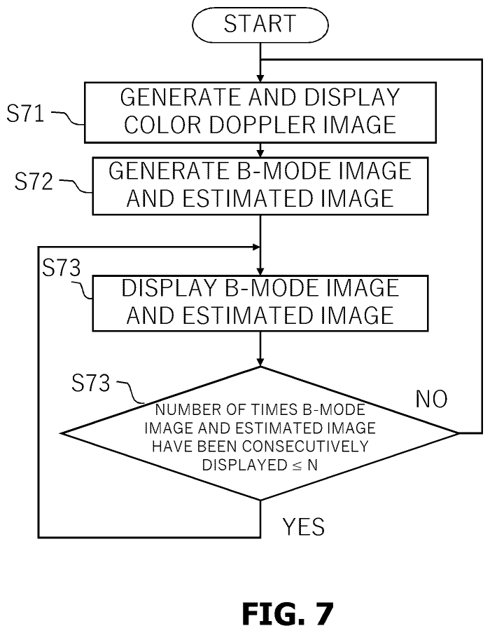

FIGS. 6A and 6B are diagrams showing a formation timing of a normal Doppler image by the Doppler processing block 204 and a formation timing of a pseudo-Doppler image by the estimation calculating block 205. FIG. 6A represents an example of the first display mode in which a display image is updated using only a normal Doppler image and FIG. 6B represents an example of the second display mode in which a display image is updated using both a normal Doppler image and a pseudo-Doppler image. In addition, FIG. 7 is a flow chart of image formation and display according to the second display mode shown in FIG. 6B.

FIG. 6A shows timings of generation and display of an image by Doppler processing. CFM1 to CFM4 denote times required for generating a B-mode image from a received signal for B-mode image generation, calculating blood flow information from a received signal for Doppler image generation, superimposing the B-mode image, and displaying a color Doppler image. In this case, four color Doppler images are to be output.

Hereinafter, a description of the second display mode will be given with reference to the flow chart shown in FIG. 7. The apparatus is switched to a control mode shown in the flow chart according to an instruction from the user, a default setting of the apparatus, or a field of medicine or a user ID. It should be noted that the processing shown in FIG. 7 is realized as the respective units 101 to 108 of the ultrasonic diagnostic apparatus 1 operate under control of the system control block 109.

In step S71, acquisition of a received signal for B-mode image generation and acquisition of a received signal for Doppler image generation are performed, one frame's worth of normal Doppler image data (color Doppler image data) is generated, and the generated normal Doppler image is displayed on the display apparatus 108. A time required by the operation is denoted by CFM1 in FIG. 6B. It should be noted that the system control block 109 has a frame memory and is capable of temporarily saving display image data that is output from the received signal processing block 106.

In step S72, a received signal for B-mode image generation of a next frame is acquired, a plurality of frames'worth of a received signal for B-mode image generation is input to the estimation calculating block 205 together with a received signal of a previous frame, and estimated blood flow information data is estimated. A time required by the operation is denoted by B1 in FIG. 6B.

In step S73, the system control block 109 updates a display image based on a pseudo-Doppler image obtained by superimposing the estimated blood flow information data (an estimated image) on the newly acquired B-mode image. For example, the system control block 109 may generate a new display image by combining the last display image and the present estimated image with a prescribed weight. Alternatively, the system control block 109 may adopt the present pseudo-Doppler image as the new display image as-is (it can be considered that a weight of the last display image is 0 and a weight of the present estimated image is 1).

In step S74, the system control block 109 checks whether or not the number of times an estimation calculation of blood flow information has been consecutively executed and display based on an estimated image has been consecutively performed has reached a prescribed number of times N (in the present example, it is assumed that N=10). When the number of times is smaller than N, a return is made to step S72. In addition, the acquisition of a received signal for B-mode image generation, estimation of blood flow information using the acquired received signal, and display of a pseudo-Doppler image are repeated until the prescribed number of times N is reached. A time required by each operation is denoted by B2 to B10 in FIG. 6B. Once the number of times an estimation calculation of blood flow information has been consecutively executed and display based on an estimated image has been consecutively performed reaches the prescribed number of times N, a return is made to step S71 and acquisition of a received signal for normal Doppler image generation and generation of color Doppler image data based on the acquired received signal are performed.

As described above, in the present display mode, processing that involves updating a display image based on a normal Doppler image and then consecutively updating a display image based on a pseudo-Doppler image a prescribed number of times is repeated.

According to the control described above, every time one frame's worth of a received signal for B-mode image generation is acquired, acquisition and display of a new pseudo-Doppler image can be performed. Therefore, image display can be realized at a higher frame rate than when updating a display image using only a normal color Doppler image. As is apparent from a comparison between FIG. 6A (a display mode in which only a normal Doppler image is used) and FIG. 6B (a display mode in which a normal Doppler image and an estimated image are used), it is shown that a larger number of frames can be displayed per unit time in the latter case.

Next, control in a case where an instruction to save a still image or a moving image is issued by the user during an imaging operation will be described. When receiving an instruction to save a still image, the system control block 109 may save both of or one of a Doppler image and an estimated image acquired at a time point that is closest to a timing at which the instruction had been received. For example, when an instruction to save a still image is input to the system control block 109 through a GUI or the like at a timing t1 shown in FIG. 6B, the Doppler image acquired at time CFM1 and the estimated image acquired at time B1 are saved. In this case, the two images may be presented to the user as candidates to be saved and the user may be asked to select an actual image to be saved. In addition, for example, when an instruction to save a still image is input at a timing t2, the Doppler image acquired at time CFM2 and the estimated image (estimated blood flow information data) acquired at time B2 are saved. With respect to the images to be saved, a setting that causes only color Doppler images to be saved or only estimated images to be saved can be separately configured as an option of the system. Furthermore, when a save instruction is issued, the flow chart shown in FIG. 7 may be interrupted to perform control for imaging a color Doppler image and the obtained image may be saved.

In addition, with respect to saving a moving image, a color Doppler image and an estimated image may be saved separately or saved in a mixed manner. Switching between these save methods can also be set as an option of the system. Furthermore, since a frame rate of an image changes depending on control in the present embodiment, when saving a moving image, interpolation and processing may be applied so as to create data at constant time intervals and a moving image with a constant frame rate may be subsequently saved.

Furthermore, while the number of times N an estimated image is consecutively displayed is a fixed value in the present embodiment, the system control block 109 may enable the number of times N to be interactively changed by the user using a GUI.

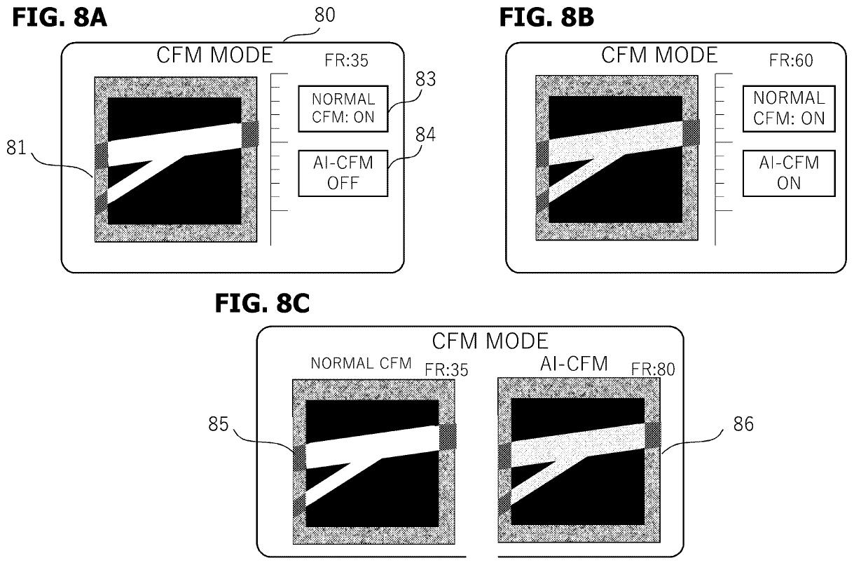

FIGS. 8A to 8C schematically show a display example of an image on the display apparatus 108. A display screen 80 includes an image display region 81, a frame rate display region 82, an indicator 83 indicating whether display of a color Doppler image is on/off, and an indicator 84 indicating whether display of an estimated image is on/off.

FIG. 8A shows a display example in a mode in which only a color Doppler image created by Doppler processing is displayed. This display mode corresponds to the mode shown in FIG. 6A. A frame rate (FR) is set to 35 fps. Since a color Doppler image is being displayed, the indicator 83 displays “Normal CFM:ON”, and since an estimated image is not displayed, the indicator 84 displays “AI-CFM:OFF”.

FIG. 8B shows a display example in a mode in which both a color Doppler image and an estimated image are displayed. This display mode corresponds to the mode shown in FIG. 5B. A frame rate is set to 60 fps. As described earlier, also including an estimated image in the display increases the frame rate as compared to a case where only a color Doppler image is displayed. In the present embodiment, while the indicator 83 displays “Normal CFM:ON” in a similar manner to FIG. 8A, in the present mode, the indicator 84 displays “AI-CFM:ON”. Accordingly, the fact that an estimated image having been estimated by the estimation calculating block 205 is included in a display image can be clearly indicated to the user. While the indicator 84 in the present embodiment notifies that an estimated image is to be displayed by character display, display of the estimated image may be notified by other systems. For example, methods such as changing a color of an outer edge of a display image or a display region, causing the outer edge to blink, and changing a color, chroma, or a pattern of a background of the display image or the display region may be adopted.

FIG. 8C is an example in which a color Doppler image and an estimated image are displayed side by side. A color Doppler image is displayed on a left side of a screen at a frame rate of 35 fps, and an estimated image is displayed on a right side of the screen at a frame rate of 80 fps. Using this display screen enables the user to check an estimated image and a correct answer image at the same time. Such a display screen is useful when evaluating or checking accuracy and reliability of the estimation calculating block 205.

### Second Embodiment

Next, another embodiment of the present invention will be described. In the present embodiment, a part of a received signal for generating a Doppler image is used to estimate blood flow information.

An overall configuration of the ultrasonic diagnostic apparatus 1 is similar to that of the first embodiment (FIG. 1). A flow from inputting a received signal for B-mode image generation and a received signal for Doppler image generation to the received signal processing block 106 up to saving the received signals in the signal storage block 202 is similar to that of the first embodiment.

In the first embodiment, a plurality of frames'worth of a received signal for B-mode image generation is used as input to the estimation calculating block 205. In the second embodiment, the input to the estimation calculating block 205 is a plurality of frames'worth of a received signal for B-mode image generation and a part of a received signal for Doppler image generation or only a part of the received signal for Doppler image generation. A part of the received signal for Doppler image generation refers to, for example, a received signal that is obtained by a part of scans (for example, one scan) when an observation region is alternately scanned a plurality of times for the purpose of Doppler image generation.

In the present embodiment, as input data of learning data to be used for learning of the learned model 305, data similar to the input data to the estimation calculating block 205 is used. In other words, in the present embodiment, learning is performed using learning data that includes, as input data, a plurality of frames'worth of a received signal for B-mode image generation and a part of a received signal for Doppler image generation or only a part of the received signal for Doppler image generation.

According to the present embodiment, since data used as a basis for obtaining an amount of Doppler shift that is calculated by the color Doppler method is to be used in estimation, estimation accuracy of blood flow information is expected to increase. In the present embodiment, although a frame rate slightly decreases from that of the first embodiment because a part of acquisition of a received signal for Doppler image generation must be performed in order to acquire an estimated image, the frame rate is higher than a case where only a color Doppler image is displayed. In addition, when alternately scanning an observation region, the fact that an estimated image can be acquired from a received signal obtained by each scan has a large effect in improving the frame rate.

### Third Embodiment

Next, yet another embodiment of the present invention will be described. While a transmission ultrasonic wave for B-mode image generation in the first and second embodiments is a convergent beam, in the present embodiment, a plane wave or a diffuse wave is used as a transmission ultrasonic wave. Due to the transmission electrical circuit 104 applying a voltage signal to the plurality of transducers 101 without imparting a time difference, an ultrasonic wave that is a plane wave or a diffuse wave is transmitted from the transducers 101.

In the present embodiment, the estimation calculating block 205 estimates blood flow information data from a plurality of frames'worth of a received signal obtained by the transmission of a plane wave or a diffuse wave. Therefore, learning of the learned model 305 uses learning data having the plurality of frames'worth of a received signal obtained by the transmission of a plane wave or a diffuse wave from the ultrasonic probe 102 as input data and blood flow information data obtained by the CFM method as correct answer data.

When using a plane wave or a diffuse wave, since information on an imaging region can be acquired by a very small number of transmissions ranging from one to several times, the frame rate can be significantly improved from a case where a B-mode image is generated by scanning with a converged ultrasonic beam. In addition, when calculating an amount of Doppler shift in the color Doppler method, transmission/reception of an ultrasonic wave is performed a plurality of times on a same scan line. Therefore, as compared to transmission/reception of a convergent beam, transmission/reception of a plane wave or a diffuse wave enables a received signal to be acquired on a same scan line at a frame rate that is closer to that of the color Doppler method. By using, in estimation, a received signal due to transmission/reception of a plane wave or a diffuse wave having a higher frame rate than a received signal for B-mode image generation as described above, an increase in estimation accuracy of blood flow information is expected.

### Fourth Embodiment

While the estimation calculating block 205 only has one learning model in the embodiments described above, the estimation calculating block 205 may have a plurality of learning models each having performed different learning. While input data of the learning data used in the learning of the plurality of learning models is similar to the learning data described above, correct answer data of the learning data is blood flow information (a Doppler image) acquired under different conditions in accordance with the learning model. Examples of different conditions include respective settings of transmission control and reception control suitable for acquiring blood flow information of an ultra low-velocity blood flow, a normal-velocity blood flow, and a high-velocity blood flow. In addition, a single learning model may be learned so as to estimate blood flow information acquired under a plurality of different conditions as described above.

According to the present embodiment, respective pieces of blood flow information of an ultra low-velocity blood flow, a normal-velocity blood flow, and a high-velocity blood flow are acquired from a received signal for B-mode image generation. Displaying these pieces of blood flow information by superimposing the information on a B-mode image enables blood flow information of a wide velocity range to be visualized at the same time.

### Other Embodiments

The embodiments described above merely represent specific examples of the present invention. A scope of the present invention is not limited to the configurations of the embodiments described above and various embodiments can be adopted without departing from the spirit of the invention.

For example, while a color Doppler image is generated and displayed in the first to fourth embodiments, only an estimated image (estimated blood flow information data) may be estimated and displayed without generating and displaying a color Doppler image. Accordingly, an image equivalent to a color Doppler method can be obtained without causing a drop in a frame rate due to Doppler processing. In addition, the Doppler processing block 204 can be omitted from the ultrasonic diagnostic apparatus 1.

In addition, while a plurality of frames'worth of a received signal for B-mode image generation is used as input data to a learned model in the first to fourth embodiments, alternatively, one frames'worth of a received signal for B-mode image generation may be used as input data to a learned model. Estimation of blood flow information can be performed and the advantageous effects of the present invention can be obtained even from one frames'worth of a received signal. Similar advantageous effects can be produced when using B-mode image data instead of a received signal as input data.

Furthermore, in the first to fourth embodiments, a learning model that uses a signal after phasing addition and quadrature detection as input and outputs blood flow information data is used when performing learning. However, the input data to the learned model may be image data after being input to the B-mode processing block. In this case, a color Doppler image having been subject to Doppler processing may be used as correct answer data. The advantageous effects of the present invention can be obtained even through such learning.

Furthermore, the disclosed technique can take the form of an embodiment of, for example, a system, an apparatus, a method, a program, or a recording medium (a storage medium). Specifically, the disclosed technique may be applied to a system constituted by a plurality of devices (for example, a host computer, an interface device, an imaging apparatus, and a web application) or to an apparatus constituted by a single device.

It is needless to say that the object of the present invention can be realized by performing the following. A recording medium (or a storage medium) on which is recorded a program code (a computer program) of software that realizes functions of the embodiments described above is supplied to a system or an apparatus. It is needless to say that the storage medium is a computer-readable storage medium. In addition, a computer (or a CPU or an MPU) of the system or the apparatus reads and executes the program code stored in the recording medium. In this case, the program code itself having been read from the recording medium is to realize the functions of the embodiments described above and the recording medium on which the program code is recorded is to constitute the present invention.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An ultrasonic diagnostic apparatus, comprising:
an ultrasonic probe configured to transmit and receive ultrasonic waves to and from an object; and
an estimation calculating unit configured to estimate data based on blood flow information from third data based on a received signal for image generation received by the ultrasonic probe by using a model having been machine-learned from learning data including first data based on a received signal for image generation that is obtained from an observation region and second data based on blood flow information of the observation region.
